(19) United States
(12) Patent Application Publication
(10) Pub. No.: US 2014/0275237 A1
(43) Pub. Date: Sep. 18, 2014

(54) BERAPROST ISOMER AS AN AGENT FOR THE TREATMENT OF VIRAL INFECTION

(71) Applicant: Gemmus Pharma Inc., San Francisco, CA (US)

(72) Inventors: DARYL H. FAULDS, Millbrae, CA (US); WILLIAM J. GUILFORD, Belmont, CA (US)

(73) Assignee: Gemmus Pharma Inc., San Francisco, CA (US)

(21) Appl. No.: 14/207,024

(22) Filed: Mar. 12, 2014

Related U.S. Application Data
(60) Provisional application No. 61/798,832, filed on Mar. 15, 2013.

(51) Int. Cl. A61K 31/343

(52) U.S. Cl. CPC A61K 31/343
USPC 514/468; 549/458

(57) ABSTRACT

In various embodiments the use of single isomer of beraprost as a therapeutic for the treatment of viral disease and other pathologies associated with the induction of a cytokine storm, such as influenza A viruses and the SARS-causing coronvirus and mutations thereof is provided.

### CROSS-REFERENCE TO RELATED APPLICATIONS

This application claims benefit of and priority to U.S. Ser. No. 61/798,832, filed on Mar. 15, 2013, which is incorporated herein by reference in its entirety for all purposes.

### STATEMENT OF GOVERNMENTAL SUPPORT

[Not Applicable]

### BACKGROUND

The influenza A virus is considered to be one of the greatest infectious disease risks to human health and any serotype is a potential agent in a catastrophic pandemic. This assessment is based on the severity and high mortality rate of the viral infection in birds and similarity to the global influenza pandemic of 1918. Public health approaches as vaccination have the disadvantage of being slow to develop and specific to individual serotype(s), which may be unsuitable against a rapidly mutating virus. Although the current anti-viral therapy is effective, resistant serotypes have been observed. We see the need for a novel treatment which can reduce the mortality of the disease by modifying an infected individual's response to the viral infection.

Clinical studies have attributed the lethality of the virus to the induction of a ‘cytokine storm’ in which the healthy individual's immune system is activated and releases large amounts of the pro-inflammatory cytokines such as INF-γ, CCL2 and IL-6. We hypothesized that a compound which inhibits INF-γ, CCL2 and IL-6 induced by dsRNA (the replicative form of influenza genetic material) should be beneficial as a stand-alone or adjunctive therapy for influenza infection.

Both seasonal and pandemic strains of the influenza viruses infect humans and cause severe disease and death amongst humans. The severity of disease has been attributed to the ability of the virus subtype to induce a potent inflammatory response which has been characterized as a hypercytokinemia. (Chan et al. (2005) Resp. Res., 6:135).

The normal response by the body to fight off a viral infection is to increase the production of inflammatory cytokines, such as interferon gamma (IFN-γ), which promote the development of T-helper type 1 (Th1) cells. In severe cases of the flu or other influenza-like-illnesses (ILI), the hyper-induction of cytokines and/or chemokines, hypercytokinemia, can lead to a prolonged inflammatory response which can cause tissue damage and death. Treatment of the hypercytokinemia requires both a reduction in the concentration of cytokines released as a consequence of infection and modulation of the lymphocyte response to infection (Id.).

Prostanoids, such as prostaglandins (PG) and prostacyclins, are cyclooxygenase products derived from C20 unsaturated fatty acids. Prostaglandins have a wide variety of effects in various tissues and cells, including, relaxation and contraction of smooth muscles, modulation of neurotransmitter release, regulation of secretions and motility in the gastrointestinal tract, regulation of the transport of ions and water in kidneys, immune system regulation, bone remodeling, and regulation of platelet aggregation, degranulation, and shape. They are also involved in apoptosis, cell differentiation, and oncogenesis (Narumiya et al. (1999) Physiol. Rev. 79, 1193-1226).

Prostaglandins exert their effects through their G protein-coupled receptors (GPCR) which are located on the cell surface. Many of the prostaglandin receptors have been cloned and characterized. In the case of Prostaglandin I2 (PGI2), the wide variety of cellular effects resulting from binding to the IP prostanoid receptor. The IP receptor is Gas-coupled and IP agonists activate adenylate cyclase, resulting in an acute burst of intracellular cAMP. cAMP has multiple effects including the activation of protein kinase A, intracellular calcium release, and -activated activation of mitogen protein kinase (MAP kinase). These effects include a potent anti-inflammatory effect on a number of different cell types. The modulatory effect was associated with IP-dependent up-regulation of intracellular cAMP and down-regulation of NF-kB activity.

Increased production of cytokines triggers inflammation, a normal response by the body to help fight a virus. However, when cytokine production becomes prolonged or excessive it can inflame airways, making it hard to breathe, which in turn can result in pneumonia and acute respiratory distress; and it can injure other organs, which can result in severe life-threatening complications.

It has recently been demonstrated that all influenza A virus subtypes and other viruses which induce cytokines in primary human alveolar and bronchial epithelial cells. Levels of cytokines and chemokines are directly related to the severity of the symptoms as seen in the flu-like-symptoms induced in patients receiving interferon treatment (Heltzer et al., (2009) J. Leuko. Biol. 85:1036-1043 and deJong et al., (2007) Nature Med., 12(10): 1203-2007).

### SUMMARY

In various embodiments a therapeutic agent is provided that inhibits the release of overstimulated cytokines and chemokines, especially interferon gamma (IFN-γ). It is believed the therapeutic agent that is useful in the treatment of influenza A, diseases associated with influenza A and other viral infections that induce flu-like-symptoms (e.g., viruses that cause the severe acute respiratory syndrome (SARS)) while being well-tolerated by the patients.

It was discovered that specific GPCR agonists, such as beraprost sodium, are a potent inhibitor of the hypercytokinemia induced by viral RNA and it was determined that the activity is due to one of the four isomers found in commercially available beraprost sodium. Thus in certain embodiments, the method described herein are directed to the use of a single isomer of beraprost or the use of compositions comprising that isomer at a higher proportion than is typically found in beraprost sodium, as a therapeutic for the treatment of pathologies characterized by the production/induction of a cytokine storm. Such pathologies include, but are not limited to human respiratory diseases associated with an induction of a hypercytokinemia, such as influenza A viruses, for example H5N1 and its mutations, or a coronavirus, for example viruses which cause the severe acute respiratory syndrome (SARS).

Thus, in certain embodiments methods are provided that comprise administering to a subject in need thereof an effective amount of an GPCR receptor agonist as a single isomer (or predominant isomer) of beraprost.

In various aspects, the invention(s) contemplated herein may include, but need not be limited to, any one or more of the following embodiments:

Embodiment 1: A method of treating a pathology characterized by hypercytokinemia, said method including: administering, or causing to be administered to subject in need of such treatment and amount of a therapeutic agent effective to partially or fully suppress said hypercytokinemia.

Embodiment 2: The method of embodiment 1, wherein the partial or full suppression of said hypercytokinemia includes a reduction in the expression of IL-6.

Embodiment 3: The method according to any one of embodiments 1-2, wherein the partial or full suppression of said hypercytokinemia includes a reduction in the expression of IFN-γ.

Embodiment 4: The method according to any one of embodiments 1-3, wherein the partial or full suppression of said hypercytokinemia includes a reduction in the expression of IL-10.

Embodiment 5: The method according to any one of embodiments 1-4, wherein the partial or full suppression of said hypercytokinemia includes a reduction in the expression of CCL2.

Embodiment 6: The method according to any one of embodiments 1-5, wherein said disease is a viral disease characterized by the induction of hypercytokinemia.

Embodiment 7: The method of embodiment 58, wherein said viral disease is an influenza A infection.

Embodiment 8: The method of embodiment 2, wherein said viral disease is an H5N1 or H5N1 mutant infection.

Embodiment 9: The method of embodiment 58, wherein said viral disease is a corona virus infection.

Embodiment 10: The method of embodiment 9, wherein said viral disease is a corona virus infection that causes acute respiratory syndrome (SARS).

Embodiment 11: The method of embodiment 58, wherein said viral disease is not influenza virus.

Embodiment 12: The method of embodiment 6, wherein said is an infection with a virus selected from the group consisting of Hepatitis A virus, Hepatitis B virus, and Hepatitis C virus.

Embodiment 13: The method of embodiment 6, wherein said disease is an infection with a virus selected from the group consisting of a coronavirus, Dengue virus, and West Nile Virus.

Embodiment 14: The method according to any one of embodiments 1-5, wherein said pathology is a pathology selected from the group consisting of graft versus host disease (GVHD), adult respiratory distress syndrome (ARDS), sepsis, smallpox, hantavirus pulmonary syndrome, tularemia, and systemic inflammatory response syndrome (SIRS).

Embodiment 15: The method according to any one of embodiments 1-9, wherein said therapeutic agent includes beraprost isomer A (BPS-314d) as a higher proportion of beraprost isomers than is found in beraprost sodium (4 isomer formulation).

Embodiment 16: The method according to any one of embodiments 1-9, wherein said beraprost isomer A (BPS-314d) is present in an amount at least 1.5 times greater than the amount of any other beraprost isomers in said composition.

Embodiment 17: The method of embodiment 10, wherein said beraprost isomer A (BPS-314d) is present in an amount at least 2 times greater than the amount of any other beraprost isomers in said composition.

Embodiment 18: The method of embodiment 10, wherein said beraprost isomer A (BPS-314d) is present in an amount at least 3 times greater than the amount of any other beraprost isomers in said composition.

Embodiment 19: The method according to any one of embodiments 1-9, wherein said therapeutic agent includes predominantly no more than three isomers of beraprost.

Embodiment 20: The method of embodiment 19, wherein one of said isomers is beraprost isomer A (BPS-314d).

Embodiment 21: The method of embodiment 19, wherein said therapeutic agent includes predominantly no more than two isomers of beraprost.

Embodiment 22: The method of embodiment 21, wherein one of said isomers is beraprost isomer A (BPS-314d).

Embodiment 23: The method according to any one of embodiments 1-9, wherein said therapeutic agent includes predominantly a single isomer of beraprost.

Embodiment 24: The method of embodiment 23, wherein said isomer is beraprost isomer A (BPS-314d).

Embodiment 25: The method according to any one of embodiments 1-9, wherein said therapeutic agent includes a substantially pure isomer of beraprost.

Embodiment 26: The method of embodiment 12, wherein said isomer is beraprost isomer A (BPS-314d).

Embodiment 27: The method according to any one of embodiments 1-26, wherein said therapeutic agent is administered in conjunction with an antiviral agent.

Embodiment 28: The method of embodiment 27, wherein said therapeutic agent is administered in conjunction with an antiviral agent selected from the group consisting of oseltamivir (Tamiflu™), zanamivir (Relenza™), amantadine, and rimantadine.

Embodiment 29: The method of embodiment 28, wherein said antiviral agent is oseltamivir.

Embodiment 30: The method of embodiment 28, wherein said antiviral agent is zanamivir.

Embodiment 31: The method according to any one of embodiments 1-30, wherein said therapeutic agent is administered via a route selected from the group consisting of inhalation, transdermal, intravenous, subcutaneous, and oral administration.

Embodiment 32: The method of embodiment 15, wherein said therapeutic agent is administered in a therapeutically effective amount ranging from about 0.001 mg/day to about 1 mg/day.

Embodiment 33: The method of embodiment 32, wherein said therapeutic agent is administered in a therapeutically effective amount ranging from about 0.001 mg/day to 0.3 mg/day.

Embodiment 34: The method of embodiment 15, wherein said therapeutic agent is administered in a therapeutically effective amount ranging from about 0.1 μg/kg/day to about 300 μg/kg/day.

Embodiment 35: A method of treating a viral disease which induces hypercytokinemia in an individual in need thereof of a therapeutically effective amount of a prostacyclin analog.

Embodiment 36: A pharmaceutical formulation including: a therapeutic agent that includes beraprost isomer A (BPS-314d) as a higher proportion of berapost isomers than is found in beraprost (4 isomer formulation); and a pharmaceutically acceptable excipient or carrier.

Embodiment 37: The formulation of embodiment 18, wherein said beraprost isomer A (BPS-314d) is present in an amount at least 1.5 times greater than the amount of any other beraprost isomers in said composition.

Embodiment 38: The formulation of embodiment 37, wherein said beraprost isomer A (BPS-314d) is present in an amount at least 2 times greater than the amount of any other beraprost isomers in said composition.

Embodiment 39: The formulation of embodiment 37, wherein said beraprost isomer A (BPS-314d) is present in an amount at least 3 times greater than the amount of any other beraprost isomers in said composition.

Embodiment 40: The formulation of embodiment 18, wherein said therapeutic agent includes predominantly or contains no more than three isomers of beraprost.

Embodiment 41: The formulation of embodiment 40, wherein one of said isomers is beraprost isomer A (BPS-314d).

Embodiment 42: The formulation according to any one of embodiments 40-41, wherein said therapeutic agent includes predominantly no more than three isomers of beraprost.

Embodiment 43: The formulation according to any one of embodiments 40-41, wherein said therapeutic agent contains no more than three isomers of beraprost.

Embodiment 44: The formulation of embodiment 18, wherein said therapeutic agent includes predominantly or contains no more than two isomers of beraprost.

Embodiment 45: The formulation of embodiment 44, wherein one of said isomers is beraprost isomer A (BPS-314d).

Embodiment 46: The formulation according to any one of embodiments 44-45, wherein said therapeutic agent includes predominantly no more than two isomers of beraprost.

Embodiment 47: The formulation according to any one of embodiments 44-45, wherein said therapeutic agent contains no more than two isomers of beraprost.

Embodiment 48: The formulation of embodiment 18, wherein said therapeutic agent includes predominantly or consists of beraprost isomer A (BPS-314d).

Embodiment 49: The formulation of embodiment 48, wherein said therapeutic agent includes predominantly beraprost isomer A (BPS-314d).

Embodiment 50: The formulation of embodiment 48, wherein said said therapeutic agent consists of beraprost isomer A (BPS-314d).

Embodiment 51: The formulation of embodiment 18, wherein said therapeutic agent includes a substantially pure beraprost isomer A (BPS-314d).

Embodiment 52: The formulation according to any one of embodiments 18-51, wherein said agent formulated for administration via a route selected from the group consisting of inhalation, transdermal, intravenous, subcutaneous, vaginal, rectal, and oral administration.

Embodiment 53: The formulation according to any one of embodiments 18-80, wherein said formulation is a unit dosage formulation.

Embodiment 54: The formulation according to any one of embodiments 18-53, wherein formulation further includes an anti-viral agent.

Embodiment 55: The formulation of embodiment 54, wherein said an antiviral agent includes an agent selected from the group consisting of oseltamivir (Tamiflu™), zanamivir (Relenza™), amantadine, and rimantadine.

Embodiment 56: The formulation of embodiment 54, wherein said an antiviral agent includes oseltamivir.

Embodiment 57: The formulation of embodiment 54, wherein said an antiviral agent includes zanamivir.

Embodiment 58: A method of treating a viral disease associated with the induction of immune response comprised of large amounts of pro-inflammatory cytokines such as IFN-γ, IL-10, IL-6, and CCL2, a “cytokine storm”, in a subject in need of such treatment, said method including administering, or causing to be administered, to the subject amount of a therapeutic agent effective to partially or fully suppress said cytokine storm.

Embodiment 59: The method of embodiment 58, wherein said viral disease was initiated by an infection with the influenza A virus.

Embodiment 60: The method of embodiment 59, wherein the influenza A virus is H5N1 or a mutation thereof.

Embodiment 61: The method of embodiment 58, wherein said viral disease is a disease initiated by a coronavirus, for example the virus which cause the severe acute respiratory syndrome (SARS) or mutations thereof.

Embodiment 62: The method of embodiment 58, wherein said viral disease is influenza A virus.

Embodiment 63: The method of embodiment 58, wherein said viral disease is not influenza virus.

Embodiment 64: The method of embodiment 63, wherein said disease initiated by an infection with a virus selected from the group consisting of Hepatitis A virus, Hepatitis B virus, and Hepatitis C virus.

Embodiment 65: The method of embodiment 63, wherein said disease is a disease initiated by an infection with a virus selected from the group consisting of a coronavirus, Dengue virus, and West Nile Virus.

Embodiment 66: The method of embodiment 65, wherein said the virus is the virus that causes severe acute respiratory syndrome (SARS).

Embodiment 67: The method according to any one of embodiments 58-66, wherein said therapeutic agent includes predominantly no more than two isomers of beraprost.

Embodiment 68: The method of embodiment 67, wherein said therapeutic agent includes predominantly a single isomer of beraprost.

Embodiment 69: The method of embodiment 67, wherein said therapeutic agent includes a substantially pure isomer of beraprost.

Embodiment 70: The method according to any one of embodiments 67-69, wherein said isomer includes Beraprost sodium (2,3,3a,8b-tetrahydro-2-hydroxyl-1-(3-hydroxyl-4-methyl-1-octen-6-ynyl)-1H-cyclopenta [b]benzofuran-5-butanoic acid, sodium salt).

Embodiment 71: The method according to any one of embodiments 67-69, wherein said isomer includes, wherein said the beraprost isomer BPS-314d ([1R,2R,3aS, 8bS]-(2,3,3a,8b-tetrahydro-2-hydroxyl-1-[(3S,4S)-(3-hydroxyl-4-methyl-1-(E)-octen-6-ynyl)-1H-cyclopenta[b]benzofuran-5-butanoic acid, sodium salt).

Embodiment 72: The method according to any one of embodiments 58-71, wherein said agent is administered via a route selected from the group consisting of inhalation, transdermal, intravenous, subcutaneous, and oral administration.

Embodiment 73: The method of embodiment 72, wherein said agent is administered in a therapeutically effective amount ranging from about 0.050 mg/day to 1 mg/day.

Embodiment 74: A method of treating a viral disease which induced a ‘cytokine storm’ in an individual in need thereof of a therapeutically effective amount of a prostacyclin analog.

Embodiment 75: A therapeutic composition including a therapeutic agent wherein said therapeutic agent includes predominantly no more than two isomers of beraprost.

Embodiment 76: The composition of embodiment 75, wherein said therapeutic agent includes predominantly a single isomer of beraprost.

Embodiment 77: The composition of embodiment 75, wherein said therapeutic agent includes a substantially pure isomer of beraprost.

Embodiment 78: The composition according to any one of embodiments 75-77, wherein said isomer includes Beraprost sodium (2,3,3a,8b-tetrahydro-2-hydroxyl-1-(3-hydroxyl-4-methyl-1-octen-6-ynyl)-1H-cyclopenta[b]benzofuran-5-butanoic acid, sodium salt).

Embodiment 79: The composition according to any one of embodiments 75-77, wherein said isomer includes, wherein said the beraprost isomer BPS-314d ([1R,2R,3aS, 8bS]-(2,3,3a,8b-tetrahydro-2-hydroxyl-1-[(3S,4S)-(3-hydroxyl-4-methyl-1-(E)-octen-6-ynyl)-1H-cyclopenta[b]benzofuran-5-butanoic acid sodium salt).

Embodiment 80: The composition according to any one of embodiments 75-79, wherein said agent formulated for administration via a route selected from the group consisting of inhalation, transdermal, intravenous, subcutaneous, and oral administration.

Embodiment 81: The composition of embodiment 80, wherein said composition is a unit dosage formulation.

### DEFINITIONS

The term “treat” when used with reference to treating, e.g. a pathology or disease refers to the mitigation and/or elimination of one or more symptoms of that pathology or disease, and/or a reduction in the rate of onset of the pathology or disease, or a reduction in severity of one or more symptoms of that pathology or disease, and/or the elimination or prevention of that pathology or disease. With respect to a viral infection, the term “treat” or “treatment” can refer to a reduction (or elimination) in infectivity of the virus and/or a reduction (or elimination) in the proliferation of the virus and/or with respect to a pathology characterized by a cytokine storm (including, but not limited to viral infections), the term “treat” or “treatment” can refer to partially or fully inhibiting the cytokine storm, e.g., as determined by a reduction in the production of pro-inflammatory cytokines). With respect t

As used herein, the phrase “a subject in need thereof” refers to a subject, as described infra, that suffers from a viral infection or other pathology characterized by a cytokine storm as described herein.

The terms “subject,” “individual,” and “patient” may be used interchangeably and refer to a mammal, preferably a human or a non-human primate, but also domesticated mammals (e.g., canine or feline), laboratory mammals (e.g., mouse, rat, rabbit, hamster, guinea pig), and agricultural mammals (e.g., equine, bovine, porcine, ovine). In various embodiments, the subject can be a human (e.g., adult male, adult female, adolescent male, adolescent female, male child, female child) under the care of a physician or other health worker in a hospital, as an outpatient, or other clinical context. In certain embodiments, the subject may not be under the care or prescription of a physician or other health worker.

The phrase “cause to be administered” refers to the actions taken by a medical professional (e.g., a physician), or a person prescribing and/or controlling medical care of a subject, that control and/or determine, and/or permit the administration of the agent(s)/compound(s) at issue to the subject. Causing to be administered can involve diagnosis and/or determination of an appropriate therapeutic or prophylactic regimen, and/or prescribing particular agent(s)/compounds for a subject. Such prescribing can include, for example, drafting a prescription form, annotating a medical record, and the like. It will be recognized that in methods involving administration, “causing to be administered” is also contemplated. Thus, for example, where “ . . . administering compound X . . . ” is recited “ . . . administering compound X or causing compound X to be administered . . . ” may be contemplated.

The term “substantially pure isomer” refers to a formulation or composition wherein among various isomers of a compound a single isomer is present at 70%, or greater or at 80% or greater, or at 90% or greater, or at 95% or greater, or at 98% or greater, or at 99% or greater, or said compound or composition comprise only a single isomer of the compound.

The term “PSS” refers to “physiological saline solution”, a solution of a salt or salts that is essentially isotonic with tissue fluids or blood. PSS, as used herein refers to a 0.9 percent solution of sodium chloride. PSS is also called normal saline solution, normal salt solution, and physiological salt solution.

As used herein, “administering” refers to local and systemic administration, e.g., including enteral, parenteral, pulmonary, and topical/transdermal administration. Routes of administration for agents (e.g., beraprost isomer(s), or pharmaceutically acceptable salts or solvates of said isomer(s)) that find use in the methods described herein include, e.g., oral (per os (p.o.)) administration, nasal or inhalation administration, administration as a suppository, topical contact, transdermal delivery (e.g., via a transdermal patch), intrathecal (IT) administration, intravenous (“iv”) administration, intraperitoneal (“ip”) administration, intramuscular (“im”) administration, intralesional administration, or subcutaneous (“sc”) administration, or the implantation of a slow-release device e.g., a mini-osmotic pump, a depot formulation, etc., to a subject. Administration can be by any route including parenteral and transmucosal (e.g., oral, nasal, vaginal, rectal, or transdermal). Parenteral administration includes, e.g., intravenous, intramuscular, intra-arterial, intradermal, subcutaneous, intraperitoneal, intraventricular, ionophoretic and intracranial. Other modes of delivery include, but are not limited to, the use of liposomal formulations, intravenous infusion, transdermal patches, etc.

The terms “systemic administration” and “systemically administered” refer to a method of administering the agent(s) described herein or composition to a mammal so that the agent(s) or composition is delivered to sites in the body, including the targeted site of pharmaceutical action, via the circulatory system. Systemic administration includes, but is not limited to, oral, intranasal, rectal and parenteral (e.g., other than through the alimentary tract, such as intramuscular, intravenous, intra-arterial, transdermal and subcutaneous) administration.

The term “co-administering” or “concurrent administration” or “administering in conjunction with” when used, for example with respect to the active agent(s) described herein e.g., beraprost isomer(s) and a second active agent (e.g., an antiviral agent), refers to administration of the agent(s) and/the second active agent such that both can simultaneously achieve a physiological effect. The two agents, however, need not be administered together. In certain embodiments, administration of one agent can precede administration of the other. Simultaneous physiological effect need not necessarily require presence of both agents in the circulation at the same time. However, in certain embodiments, co-administering typically results in both agents being simultaneously present in the body (e.g., in the plasma) at a significant fraction (e.g., 20% or greater, preferably 30% or 40% or greater, more preferably 50% or 60% or greater, most preferably 70% or 80% or 90% or greater) of their maximum serum concentration for any given dose.

The term “cytokine storm”, also known as a “cytokine cascade” or “hypercytokinemia: is a potentially fatal immune reaction typically consisting of a positive feedback loop between cytokines and immune cells, with highly elevated levels of various cytokines (e.g. IFN-γ, IL-10, IL-6, CCL2, etc.).

### BRIEF DESCRIPTION OF THE DRAWINGS

FIG. 1 illustrates the four isomers that comprise beraprost.

### DETAILED DESCRIPTION

In various embodiments, the methods and compositions described herein pertain to the discovery that a single isomer of beraprost is predominantly responsible for the ability of beraprost to modulate a mammalian (e.g., a human or non-human mammal) immune response and that the three other isomers have a neutral or negative effect in the treatment or prevention of viral diseases. Accordingly, in various embodiments, compositions comprising the substantially pure isomer and the use of such compositions in the treatment and/or prophylaxis of viral diseases.

In various embodiments the isomer useful in the methods described herein is one that inhibits the release of cytokines and/or chemokines in response to a viral infection, particularly a viral infection that induces a cytokine storm. In certain embodiments the infection is one produce by the influenza A virus and/or the coronavirus, which cause the severe acute respiratory syndrome (SARS). The inhibition can be can be determined by one of skill in the art by methods known in the art or as taught herein, without undue experimentation.

In one illustrative the isomer (modulator of the immune system) is selected from the isomers of beraprost (beraprost sodium) and derivatives of the four isomers that comprise beraprost sodium. The pharmacological effects of beraprost sodium are known from U.S. Pat. No. 8,183,286. However, it was a surprising discovery that a single isomer of beraprost is the major factor (e.g., provides most of the observed activity) in moderating the immune system and the other three isomers were determined to have a neutral or negative effect on the immune system. It is believed that a single isomer of beraprost has not been previously described as being effective in the treatment or prevention of viral diseases.

Accordingly, in an illustrative embodiment, a beraprost isomer useful in treating viral infections according to the present invention is one of the four isomers of beraprost sodium (2,3,3a,8b-tetrahydro-2-hydroxyl-1-(3-hydroxyl-4-methyl-1-octen-6-ynyl)-1H-cyclopenta[b]benzofuran-5-butanoic acid, sodium salt). Beraprost sodium is a mixture of four isomers, two diastereomers (BPS-314 and BPS-315) and their enantiomers which are BPS-314d and BPS-3141 and BPS-315d and BPS-315/(FIG. 1). These isomers are referred to herein as isomers A, B, C, and D as shown in Table 1.

TABLE 1
ISOMERS of beraprost.

| Isomer | As shown in Fig. 1 |
|---|---|
| Isomer A | BPS-314d |
| Isomer B | BPS-3151 |
| Isomer C | BPS-315d |
| Isomer D | BPS-3141 |

It was discovered that isomer A (BPS-314d) predominantly accounts for the immune-modulating activity of beraprost, while isomers B, D, and D have a neutral or negative effect. Accordingly it is believed that compositions comprising substantially pure isomer A or comprising an increased amount of isomer A while decreasing the percentages of isomer B, and/or isomer C, and/or isomer D can be effectively used to treat pathologies characterized by a cytokine storm.

The cytokine storm is a potentially fatal immune reaction typically consisting of a positive feedback loop between cytokines and immune cells, with highly elevated levels of various cytokines (e.g. IFN-γ, IL-10, IL-6, CCL2, etc.). Cytokine storms can occur in a number of infectious and non-infectious diseases. Such disease include, but are not limited to, graft versus host disease (GVHD), adult respiratory distress syndrome (ARDS), sepsis, avian influenza, smallpox, hantavirus pulmonary syndrome, tularemia, severe cases of leptospirosis, and systemic inflammatory response syndrome (SIRS). In certain embodiments, the use of the therapeutic compositions and/or pharmaceutical formulations described herein in the treatment and/or prophylaxis of any of these pathologies and especially in the treatment of viral infections (e.g., influenza infection) is contemplated.

### Beraprost Isomer(s).

It was discovered that isomer A (BPS-314d) predominantly accounts for the immune-modulating activity of beraprost, while isomers B, D, and D have a neutral or negative effect. Accordingly it is believed that compositions comprising substantially pure isomer A or comprising an increased amount of isomer A while decreasing the percentages of isomer B, and/or isomer C, and/or isomer D can be effectively used to treat pathologies characterized by a cytokine storm. Accordingly, in various embodiments, therapeutic compositions comprising combinations and/or percentages of beraprost isomers that differ from that found in beraprost sodium are contemplated.

In certain embodiments, therapeutic compositions comprising beraprost isomer A (BPS-314d) as a higher proportion of berapost isomers than is found in beraprost sodium (4 isomer formulation) are contemplated. In certain embodiments the beraprost isomer A (BPS-314d) is present in an amount at least 1.2 times greater, or at least 1.5 times greater, or at least 2 times greater, or at least 2.5 times greater, or at least 3 times greater, or at least 3.5 times greater, or at least 4 times greater, or at least 5 times greater, or at least 10 or 15, or 20 times greater than the amount of any other beraprost isomers in the composition. In certain embodiments the therapeutic agent comprises predominantly or contains no more than three isomers of beraprost, where typically one of the isomers is beraprost isomer A (BPS-314d). In certain embodiments the therapeutic agent comprises predominantly or contains no more than two isomers of beraprost, where typically one of the two isomers is beraprost isomer A (BPS-314d). in certain embodiments the therapeutic agent comprises predominantly or consists of beraprost isomer A (BPS-314d), and in certain embodiments the therapeutic agent comprises or consists of a substantially pure beraprost isomer A (BPS-314d).

### Pharmaceutical Formulations.

The pharmacologically active beraprost isomers identified herein useful in the methods described (e.g., in the treatment of a pathology associated with a cytokine storm (such as a viral infection, e.g. influenza infection) herein can be processed in accordance with conventional methods of galenic pharmacy to produce medicinal agents for treating diseases associated with viral infections. In certain embodiments compositions comprising an active beraprost isomer described herein are administered to a mammal in need thereof, e.g., to a mammal at risk for or infected with influenza of a non-influenza virus that produces influenza-like symptoms. The pharmaceutical compositions comprise the beraprost isomer(s) in an effective amount (in an amount effective to treat the pathology, e.g., an amount effective to treat a viral infection (e.g., an influenza infection) and/or to inhibit a cytokine storm) and one or more pharmaceutically acceptable carriers/excipients.

The active agent(s) (beraprost isomer(s) can be administered in the “native” form or, if desired, in the form of salts, esters, amides, clathrates, prodrugs, derivatives, and the like, provided the salt, ester, amide, clathrate, prodrug or derivative is suitable pharmacologically, i.e., effective in the present method(s). Salts, esters, amides, prodrugs and other derivatives of the active agents can be prepared using standard procedures known to those skilled in the art of synthetic organic chemistry and described, for example, by March (1992) Advanced Organic Chemistry; Reactions, Mechanisms and Structure, 4th Ed. N.Y. Wiley-Interscience.

Methods of formulating such derivatives are known to those of skill in the art. For example, a pharmaceutically acceptable salt can be prepared for any compound described herein having a functionality capable of forming a salt. A pharmaceutically acceptable salt is any salt that retains the activity of the parent compound and does not impart any deleterious or untoward effect on the subject to which it is administered and in the context in which it is administered.

In various embodiments pharmaceutically acceptable salts may be derived from organic or inorganic bases. The salt may be a mono or polyvalent ion. Of particular interest are the inorganic ions, lithium, sodium, potassium, calcium, and magnesium. Organic salts may be made with amines, particularly ammonium salts such as mono-, di- and trialkyl amines or ethanol amines. Salts may also be formed with caffeine, tromethamine and similar molecules.

Methods of formulating pharmaceutically active agents as salts, esters, amides, clathrates, prodrugs, and the like are well known to those of skill in the art. For example, salts can be prepared from the free base using conventional methodology that typically involves reaction with a suitable acid. Generally, the base form of the drug is dissolved in a polar organic solvent such as methanol or ethanol and the acid is added thereto. The resulting salt either precipitates or can be brought out of solution by addition of a less polar solvent. Suitable acids for preparing acid addition salts include, but are not limited to both organic acids, e.g., acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like, as well as inorganic acids, e.g., hydrochloric acid, hydrobromic acid, sulfuric acid, nitric acid, phosphoric acid, and the like. An acid addition salt can be reconverted to the free base by treatment with a suitable base. Certain particularly preferred acid addition salts of the active agents herein include halide salts, such as may be prepared using hydrochloric or hydrobromic acids. Conversely, preparation of basic salts of the active agents of this invention are prepared in a similar manner using a pharmaceutically acceptable base such as sodium hydroxide, potassium hydroxide, ammonium hydroxide, calcium hydroxide, trimethylamine, or the like. Particularly preferred basic salts include alkali metal salts, e.g., the sodium salt, and copper salts.

In certain embodiments for the preparation of salt forms of basic drugs, the pKa of the counterion is preferably at least about 2 pH units lower than the pKa of the drug. Similarly, for the preparation of salt forms of acidic drugs, the pKa of the counterion is preferably at least about 2 pH units higher than the pKa of the drug. This permits the counterion to bring the solution's pH to a level lower than the pHₘₐₓ to reach the salt plateau, at which the solubility of salt prevails over the solubility of free acid or base. The generalized rule of difference in pKa units of the ionizable group in the active pharmaceutical ingredient (API) and in the acid or base is meant to make the proton transfer energetically favorable. When the pKa of the API and counterion are not significantly different, a solid complex may form but may rapidly disproportionate (i.e., break down into the individual entities of drug and counterion) in an aqueous environment.

Typically, the counterion is a pharmaceutically acceptable counterion. Suitable anionic salt forms include, but are not limited to acetate, benzoate, benzylate, bitartrate, bromide, carbonate, chloride, citrate, edetate, edisylate, estolate, fumarate, gluceptate, gluconate, hydrobromide, hydrochloride, iodide, lactate, lactobionate, malate, maleate, mandelate, mesylate, methyl bromide, methyl sulfate, mucate, napsylate, nitrate, pamoate (embonate), phosphate and diphosphate, salicylate and disalicylate, stearate, succinate, sulfate, tartrate, tosylate, triethiodide, valerate, and the like, while suitable cationic salt forms include, but are not limited to aluminum, benzathine, calcium, ethylene diamine, lysine, magnesium, meglumine, potassium, procaine, sodium, tromethamine, zinc, and the like.

In certain embodiments the active agents (e.g., beraprost isomer(s)) are formulated as a sodium salt.

Preparation of esters typically involves functionalization of hydroxyl and/or carboxyl groups that are present within the molecular structure of the active agent. In certain embodiments, the esters are typically acyl-substituted derivatives of free alcohol groups, i.e., moieties that are derived from carboxylic acids of the formula RCOOH where R is alky, and preferably is lower alkyl. Esters can be reconverted to the free acids, if desired, by using conventional hydrogenolysis or hydrolysis procedures.

Amides can also be prepared using techniques known to those skilled in the art or described in the pertinent literature. For example, amides may be prepared from esters, using suitable amine reactants, or they may be prepared from an anhydride or an acid chloride by reaction with ammonia or a lower alkyl amine.

In various embodiments, the active agents identified herein are useful for parenteral, topical, oral, nasal (or otherwise inhaled), rectal, or local administration, such as by aerosol or transdermally, for prophylactic and/or therapeutic treatment of one or more of the pathologies/indications described herein (e.g., various viral infections associated with a cytokine cascade, non-viral pathologies associated with a cytokine cascade, and the like).

The active agents described herein can also be combined with a pharmaceutically acceptable carrier (excipient) to form a pharmacological composition. Pharmaceutically acceptable carriers can contain one or more physiologically acceptable compound(s) that act, for example, to stabilize the composition or to increase or decrease the absorption of the active agent(s). Physiologically acceptable compounds can include, for example, carbohydrates, such as glucose, sucrose, or dextrans, antioxidants, such as ascorbic acid or glutathione, chelating agents, low molecular weight peptides, protection and uptake enhancers such as lipids, compositions that reduce the clearance or hydrolysis of the active agents, or excipients or other stabilizers and/or buffers.

Other physiologically acceptable compounds, particularly of use in the preparation of tablets, capsules, gel caps, and the like include, but are not limited to binders, diluent/fillers, disentegrants, lubricants, suspending agents, and the like.

In certain embodiments, to manufacture an oral dosage form (e.g., a tablet), an excipient (e.g., lactose, sucrose, starch, mannitol, etc.), an optional disintegrator (e.g. calcium carbonate, carboxymethylcellulose calcium, sodium starch glycollate, crospovidone etc.), a binder (e.g. alpha-starch, gum arabic, microcrystalline cellulose, carboxymethylcellulose, polyvinylpyrrolidone, hydroxypropylcellulose, cyclodextrin, etc.), and an optional lubricant (e.g., talc, magnesium stearate, polyethylene glycol 6000, etc.), for instance, are added to the active component or components (e.g., EP4 agonist(s)) and the resulting composition is compressed. Where necessary the compressed product is coated, e.g., known methods for masking the taste or for enteric dissolution or sustained release. Suitable coating materials include, but are not limited to ethyl-cellulose, hydroxymethylcellulose, polyoxyethylene glycol, cellulose acetate phthalate, hydroxypropylmethylcellulose phthalate, and Eudragit (Rohm & Haas, Germany; methacrylic-acrylic copolymer).

Other physiologically acceptable compounds include wetting agents, emulsifying agents, dispersing agents or preservatives that are particularly useful for preventing the growth or action of microorganisms. Various preservatives are well known and include, for example, phenol and ascorbic acid. One skilled in the art would appreciate that the choice of pharmaceutically acceptable carrier(s), including a physiologically acceptable compound depends, for example, on the route of administration of the active agent(s) and on the particular physio-chemical characteristics of the active agent(s).

In certain embodiments the excipients are sterile and generally free of undesirable matter. These compositions can be sterilized by conventional, well-known sterilization techniques. For various oral dosage form excipients such as tablets and capsules sterility is not required. The USP/NF standard is usually sufficient.

The pharmaceutical compositions can be administered in a variety of unit dosage forms depending upon the method of administration. Suitable unit dosage forms, include, but are not limited to powders, tablets, pills, capsules, lozenges, suppositories, patches, nasal sprays, injectibles, implantable sustained-release formulations, mucoadherent films, topical varnishes, lipid complexes, etc.

Pharmaceutical compositions comprising the active agents (e.g., EP4 agonists) described herein can be manufactured by means of conventional mixing, dissolving, granulating, dragee-making, levigating, emulsifying, encapsulating, entrapping or lyophilizing processes. Pharmaceutical compositions can be formulated in a conventional manner using one or more physiologically acceptable carriers, diluents, excipients or auxiliaries that facilitate processing of the active agent into preparations that can be used pharmaceutically. Proper formulation is dependent upon the route of administration chosen.

For topical administration the active agent(s) described herein may be formulated as solutions, gels, ointments, creams, suspensions, and the like as are well-known in the art. Systemic formulations include, but are not limited to, those designed for administration by injection, e.g. subcutaneous, intravenous, intramuscular, intrathecal or intraperitoneal injection, as well as those designed for transdermal, transmucosal oral or pulmonary administration. For injection, the active agents described herein can be formulated in aqueous solutions, preferably in physiologically compatible buffers such as Hanks solution, Ringer's solution, or physiological saline buffer and/or in certain emulsion formulations. The solution can contain formulatory agents such as suspending, stabilizing and/or dispersing agents. In certain embodiments the active agent(s) can be provided in powder form for constitution with a suitable vehicle, e.g., sterile pyrogen-free water, before use. For transmucosal administration, penetrants appropriate to the barrier to be permeated can be used in the formulation. Such penetrants are generally known in the art.

For oral administration, the formulations can involve combining the active agent(s) with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. For oral solid formulations such as, for example, powders, capsules and tablets, suitable excipients include fillers such as sugars, such as lactose, sucrose, mannitol and sorbitol; cellulose preparations such as maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP); granulating agents; and binding agents. If desired, disintegrating agents may be added, such as the cross-linked polyvinylpyrrolidone, agar, or alginic acid or a salt thereof such as sodium alginate. If desired, solid dosage forms may be sugar-coated or enteric-coated using standard techniques.

For oral liquid preparations such as, for example, suspensions, elixirs and solutions, suitable carriers, excipients or diluents include water, glycols, oils, alcohols, etc. Additionally, flavoring agents, preservatives, coloring agents and the like can be added. For buccal administration, the compositions may take the form of tablets, lozenges, etc. formulated in conventional manner.

For administration by inhalation, the active agent(s) (e.g., EP4 agonists) are conveniently delivered in the form of an aerosol spray from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

In various embodiments the active agent(s) can be formulated in rectal or vaginal compositions such as suppositories or retention enemas, e.g., containing conventional suppository bases such as cocoa butter or other glycerides.

In addition to the formulations described previously, the compounds may also be formulated as a depot preparation. Such long acting formulations can be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

Alternatively, other pharmaceutical delivery systems can be employed. Liposomes and emulsions are well known examples of delivery vehicles that may be used to protect and deliver pharmaceutically active compounds. Certain organic solvents such as dimethylsulfoxide also can be employed, although usually at the cost of greater toxicity.

Additionally, the compounds may be delivered using a sustained-release system, such as semipermeable matrices of solid polymers containing the therapeutic agent. Various uses of sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for compound stabilization may be employed.

In certain embodiments, the active agents described herein are administered orally. This is readily accomplished by the use of tablets, caplets, lozenges, liquids, and the like.

In certain embodiments the active agents described herein are administered systemically (e.g., orally, or as an injectable) in accordance with standard methods well known to those of skill in the art. In other preferred embodiments, the agents can also be delivered through the skin using conventional transdermal drug delivery systems, i.e., transdermal “patches” wherein the active agent(s) are typically contained within a laminated structure that serves as a drug delivery device to be affixed to the skin. In such a structure, the drug composition is typically contained in a layer, or “reservoir,” underlying an upper backing layer. It will be appreciated that the term “reservoir” in this context refers to a quantity of “active ingredient(s)” that is ultimately available for delivery to the surface of the skin. Thus, for example, the “reservoir” may include the active ingredient(s) in an adhesive on a backing layer of the patch, or in any of a variety of different matrix formulations known to those of skill in the art. The patch may contain a single reservoir, or it may contain multiple reservoirs.

In one illustrative embodiment, the reservoir comprises a polymeric matrix of a pharmaceutically acceptable contact adhesive material that serves to affix the system to the skin during drug delivery. Examples of suitable skin contact adhesive materials include, but are not limited to, polyethylenes, polysiloxanes, polyisobutylenes, polyacrylates, polyurethanes, and the like. Alternatively, the drug-containing reservoir and skin contact adhesive are present as separate and distinct layers, with the adhesive underlying the reservoir which, in this case, may be either a polymeric matrix as described above, or it may be a liquid or hydrogel reservoir, or may take some other form. The backing layer in these laminates, which serves as the upper surface of the device, preferably functions as a primary structural element of the “patch” and provides the device with much of its flexibility. The material selected for the backing layer is preferably substantially impermeable to the active agent(s) and any other materials that are present.

In certain embodiments, one or more active agents described herein can be provided as a “concentrate”, e.g., in a storage container (e.g., in a premeasured volume) ready for dilution, or in a soluble capsule ready for addition to a volume of water, alcohol, hydrogen peroxide, or other diluent.

In certain embodiments the active agents described herein (e.g., beraprost isomer(s)) are preferably suitable for oral administration. In various embodiments the active agent(s) in the oral compositions can be either coated or non-coated. The preparation of enteric-coated particles is well known to those of skill in the art and various examples are provided for example in U.S. Pat. Nos. 4,786,505 and 4,853,230.

In certain embodiments the compositions used in the methods described herein comprise the desired beraprost isomer(s) in an effective amount to achieve a pharmacological effect or therapeutic improvement without undue adverse side effects. In certain embodiments, a therapeutic improvement includes but is not limited to inhibition of proinflammatory cytokines, or a cytokine cascade and/or mitigation or prevention of one or more symptoms associated with a an influenza infection or one or more flu-like symptoms associated with a non-influenza viral infection.

In certain embodiments the active ingredients of are preferably formulated in a single oral dosage form containing all active ingredients. Such oral formulations include solid and liquid forms. It is noted that solid formulations are preferred in view of the improved stability of solid formulations as compared to liquid formulations and better patient compliance.

In one illustrative embodiment, the active agents (e.g., beraprost isomer(s)) are formulated in a single solid dosage form such as multi-layered tablets, suspension tablets, effervescent tablets, powder, pellets, granules or capsules comprising multiple beads as well as a capsule within a capsule or a double chambered capsule. In another embodiment, the active agents may be formulated in a single liquid dosage form such as suspension containing all active ingredients or dry suspension to be reconstituted prior to use.

In certain embodiments the active angent(s) are formulated as enteric-coated delayed-release granules or as granules coated with non-enteric time-dependent release polymers in order to avoid contact with the gastric juice. Non-limiting examples of suitable pH-dependent enteric-coated polymers are: cellulose acetate phthalate, hydroxypropylmethylcellulose phthalate, polyvinylacetate phthalate, methacrylic acid copolymer, shellac, hydroxypropylmethylcellulose succinate, cellulose acetate trimellitate, and mixtures of any of the foregoing. A suitable commercially available enteric material, for example, is sold under the trademark Eudragit L 100-55. This coating can be spray coated onto a substrate.

Illustrative non-enteric-coated time-dependent release polymers include, for example, one or more polymers that swell in the stomach via the absorption of water from the gastric fluid, thereby increasing the size of the particles to create thick coating layer. The time-dependent release coating generally possesses erosion and/or diffusion properties that are independent of the pH of the external aqueous medium. Thus, the active ingredient is slowly released from the particles by diffusion or following slow erosion of the particles in the stomach.

Illustrative non-enteric time-dependent release coatings are for example: film-forming compounds such as cellulosic derivatives, such as methylcellulose, hydroxypropyl methylcellulose (HPMC), hydroxyethylcellulose, and/or acrylic polymers including the non-enteric forms of the Eudragit brand polymers. Other film-forming materials can be used alone or in combination with each other or with the ones listed above. These other film forming materials generally include, for example, poly(vinylpyrrolidone), Zein, poly(ethylene glycol), poly(ethylene oxide), poly(vinyl alcohol), poly(vinyl acetate), and ethyl cellulose, as well as other pharmaceutically acceptable hydrophilic and hydrophobic film-forming materials. These film-forming materials may be applied to the substrate cores using water as the vehicle or, alternatively, a solvent system. Hydro-alcoholic systems may also be employed to serve as a vehicle for film formation.

Other materials suitable for making the time-dependent release coating of the compounds described herein include, by way of example and without limitation, water soluble polysaccharide gums such as carrageenan, fucoidan, gum ghatti, tragacanth, arabinogalactan, pectin, and xanthan; water-soluble salts of polysaccharide gums such as sodium alginate, sodium tragacanthin, and sodium gum ghattate; water-soluble hydroxyalkylcellulose wherein the alkyl member is straight or branched of 1 to 7 carbons such as hydroxymethylcellulose, hydroxyethylcellulose, and hydroxypropylcellulose; synthetic water-soluble cellulose-based lamina formers such as methyl cellulose and its hydroxyalkyl methylcellulose cellulose derivatives such as a member selected from the group consisting of hydroxyethyl methylcellulose, hydroxypropyl methylcellulose, and hydroxybutyl methylcellulose; other cellulose polymers such as sodium carboxymethylcellulose; and other materials known to those of ordinary skill in the art. Other lamina forming materials that can be used for this purpose include, but are not limited to poly(vinylpyrrolidone), polyvinylalcohol, polyethylene oxide, a blend of gelatin and polyvinyl-pyrrolidone, gelatin, glucose, saccharides, povidone, copovidone, poly(vinylpyrrolidone)-poly(vinyl acetate) copolymer.

While the compositions and methods are described herein with respect to use in humans, they are also suitable for animal, e.g., veterinary use. Thus certain preferred organisms include, but are not limited to humans, non-human primates, canines, equines, felines, porcines, ungulates, largomorphs, and the like.

The foregoing formulations and administration methods are intended to be illustrative and not limiting. It will be appreciated that, using the teaching provided herein, other suitable formulations and modes of administration can be readily devised.

For treatment of a patient having a pathology characterized by a cytokine cascade (e.g., a viral infection such as influenza A infection), the dosage of the composition comprising a beraprost isomer (e.g., beraprost isomer A (BPS-314d)) will be that amount that is effective to treat the pathology such as a viral disease (the “effective amount”) and/or to partially or fully inhibit the cytokine cascade, e.g., as indicated by the production of a pro-inflammatory cytokine such as INF-γ, and/or CCL2, and/or IL-6. The effective amount of therapeutic agent may vary depending on the route of administration, the age and weight of the patient, the nature and severity of the disorder to be treated, and similar factors. The effective amount can be determined without undue experimentation by methods known to those of skill in the art. In certain embodiments, the daily dose is generally about 0.1 to about 300 μg/kg/day or to about 200 μg/kg/day, or about 1 to about 300 μg/kg/day, when administered to human patients, it being possible for the dose to be given as a single dose to be administered once or divided into two or more daily doses.

In certain embodiments beraprost may be delivered as a co-treatment together with other anti-viral or anti-inflammatory compounds, such as, but not limited to, oseltamivir (Tamiflu™) and zanamivir (Relenza™). The compounds may be delivered to the patient at the same time or sequentially as separate formulations, or they may be combined and delivered as a single formulation.

Without further elaboration, it is believed that one skilled in the art can, using the preceding description, utilize the present invention to its fullest extent. The following specific embodiments are, therefore, to be construed as illustrative, and not limiting.

### EXAMPLES

The following examples are offered to illustrate, but not to limit the claimed invention.

### Example 1

### Separation of Isomers

### Principle:

Separation of the isomers that comprise beraprost can be accomplished by the use of the chiral separation method. The two diastereomers of beraprost can be separated by normal chromatographic methods, but the separation of one of the diastereomers from its corresponding optical isomers typically requires the resolution of the isomers and a chiral column.

### Procedure:

No single column was identified which would allow for the preparative separation of all four isomers, so a two-step method was identified. On a RegisPack column peak 1, peaks 2 and 3, and peak 4 were resolved. Peaks 2 and 3 were resolved on an AD-H column. Peak numbering was based on the order of elution from a Chiral AGP column eluting with a mixture of sodium phosphate buffer (20 mM, pH=7.0) and acetonitrile (98:2).

The preparative separation was carried out using a Supercritical Fluid Chromatography (SFC) on a chiral column. Separation of 1 g of beraprost as the four component mixture was carried out in a two-step process of (1) RegisPack column (5 micron, 30×250 mm) and eluting with a mixture of methanol and carbon dioxide (20:80) at a flow rate of 80 g/min and detection at 210 nm and (2) AD-H column (5 micron, 30×250 mm) and eluting with a mixture of methanol and carbon dioxide (20:80) at a flow rate of 80 g/min and detection at 210 nm. Isomers A, B, C, and D were isolated from 1 g of beraprost in the following amounts 230 mg, 209 mg, 195 mg and 240 mg of A, B, C, and D, respectively. Compounds were analyzed by NMR spectroscopy, but assignment is based on literature president (Wakita et al. (2000) Heterocycles, 53(5):1085-1110).

### Example 2

### NMR Analysis

### Peak Assignment Based on of Isolated Isomers

### Principle:

The structure of the different isomers was carried out using NMR spectroscopic techniques. The hydrogens on each carbon were assigned to peaks in the NMR spectrum for compounds B and D.

### Procedure:

NMR spectra were obtained on each isomer using deuterated methanol as solvent. The results were correlated to NMR spectra obtained on the diastereomeric mixtures of isomers A and D, and isomers B and C using deuterated methanol and chloroform. The spectra in deuterated chloroform allowed for direct comparison with the corresponding spectra reported for isomers BPS-314 and BPS-315 (Wakita et al., supra.).

### Results:

The assignment of isomers A and D as enantiomers and isomers B and C as enantiomers was made based on their identical NMR spectra. The peak of particular importance was the peak corresponding to the hydrogens on C-18 (see, e.g., Table 2). Based on correlations with published data for the peak of Hydrogen at C-18, isomers A and D and isomers B and C correspond to BPS-314 and BPS-315.

TABLE 2
Peak assignment of beraprost isomers B and D

[chemical structure]

| Hydrogen bound to | | |
|---|---|---|
| carbon | Isomer B | Isomer D |
| 2 | 2.28 | 2.17 |
| 3 | 1.88 | 1.88 |
| 4 | 2.59 | 2.56 |
| 6 | 6.93 | 6.93 |
| 7 | 6.73 | 6.70 |
| 8 | 6.98 | 6.94 |
| 11 | 3.42 | 3.41 |
| 12 | 5.06 | 5.04 |
| 13 | 2.64 & 1.85 | 2.64 & 1.85 |
| 14 | 3.89 | 3.87 |
| 15 | 2.28 | 2.28 |
| 16 | 5.73 | 5.72 |
| 17 | 5.57 | 5.55 |
| 18 | 4.05 | 3.98 |
| 19 | 1.70 | 1.71 |
| 20 | 1.04 | 0.99 |
| 21 | 2.04 & 2.28 | 2.12 & 2.29 |

### Example 3

### Relative Activity of Beraprost Isomers in Cytokine Release Assay

### Principle:

The ability of a compound to inhibit the release of pro-inflammatory cytokines from activated human immune cells was probed. Compounds that can inhibit cytokine release should be active in the animal model of influenza.

### Procedure:

Human donor normal Peripheral Blood Mononuclear Cells (PBMCs) were obtained from AllCells (Emeryville, Calif.) through an IRB-approved donor program. Solutions of beraprost isomers and Poly r(I:C) (10 mml of a 2 mmg/mL solution) were added to the wells of a 96-well culture microplate. The fresh cells (1×10⁶ cells) were added and incubated for 18 h at 37° C., 97% relative humility and 5% carbon dioxide. The supernatant was isolated and the human TNF-alpha concentration was determined using a commercial ELISA kit. Statistical analysis was performed using Prism using a 4 parameter logistic nonlinear model.

### Results:

Inhibition of TNFalpha production using beraprost and beraprost isomers A to D in human PBMCs activated with Poly r(I:C) with Logistic model fit (Table 3).

TABLE 3
EC₅₀ values for reduction of cytokine release from human PBMCs by
individual isomers of beraprost and beraprost.

| Compound: | A | B | C | D | Beraprost |
|---|---|---|---|---|---|
| EC₅₀: | 4 nM | No | 25 nM | No | 11 nM |
| | | inhibition | | inhibition | |

### Example 4

### Demonstration of Superiority of Isomer A of Beraprost in a Mouse Lethal Challenge Model of Influenza

### Increased Survival

### Principle:

In the mouse lethal challenge influenza model, mice are exposed to lethal dose of the influenza virus. Typically infected animals die between days 4-8, with 90-100% mortality achieved by day 8 at this dose. The lungs are severely inflamed and exhibit extreme lung consolidation. Modulation of the immune system would decrease inflammation, limit lung consolidation and increase survival.

### Procedure:

In the mouse lethal challenge influenza model, mice are inoculated with virus and treatment is initiated about 4 hours later. Mice are monitored daily and number of surviving animals noted.

### Virus:

Influenza A/Duck/MN/1525/81 (H5N1) was obtained from Dr. Robert Webster of St. Jude Hospital, Memphis, Term. The virus was passaged through mice until adapted to the point of being capable of inducing pneumonia-associated death in the animals (Barnard (2009) fAntiviral Res. 82(2): A110-122.). The viral dose was 1×10⁵ CCID₅₀ administered intranasally.

### Animals:

Female 17-20 g BALB/c mice were obtained from Charles River Laboratories (Wilmington, Mass.) for this study. They were maintained on Wayne Lab Blox and tap water ad libitum. They were quarantined for 24 h prior to use.

### Experimental Design:

Groups of 15 mice were administered GP-1001 at 1.6 mg/kg/d or one of four isomers intraperitoneally (i.p.) diluted in PSS at 0.8 mg/kg/d twice a day for 10 days (bid×10) at 0 h just prior to virus exposure. Fifteen mice were given ribavirin i.p. at 75 mg/kg/d twice a day (bid) for 5 days beginning just prior to virus exposure. Doses were given 8 hours apart. In addition, 20 mice received PSS by the i.p. route using the treatment regimen described above.

### Survival Analysis:

Survival analysis was done using the Kaplan-Meier method and a Logrank test. That analysis revealed significant differences among the treatment groups. Therefore, pairwise comparisons of survivor curves (PSS vs. any treatment) were analyzed by the Gehan-Breslow-Wilcoxon test, and the relative significance was adjusted to a Bonferroni-corrected significance threshold for the number of treatment comparisons done.

### Ethics Regulation of Laboratory Animals:

This study was conducted in accordance with and with the approval of the Institutional Animal Care and Use Committee of Utah State University. The work was done in the AAALAC-accredited Laboratory Animal Research Center of Utah State University. Initial accreditation was granted on Feb. 10, 1986 and has been maintained to the present time (last renewal: Sep. 24, 2011). The Animal Welfare Assurance Number is A3801-01 and was last reviewed by the National institutes of Health on Jun. 8, 2011 in accordance to the National Institutes of Health Guide for the Care and Use of Laboratory Animals (2010 Edition) and expires on Feb. 28, 2014.

### Results:

The survival data and the mean day of death (MDD) are indicated in Table 4.

TABLE 4
Survival and mean day of death (MDD) for individual
isomers of beraprost and for beraprost.

| Compound: | PSS | A | B | C | D | Beraprost |
|---|---|---|---|---|---|---|
| MDD | 10 | 13 | 7 | 8 | 7 | 11.5 |
| Survival | 1/19 | 4/10 | 0/10 | 0/10 | 0/10 | 3/10 |

### Example 5

### Demonstration of Superiority of Isomer A of Beraprost in a Mouse Lethal Challenge Model of Influenza

### Decrease in Mouse Weight at Day 6 after Infection

### Principle:

The weight of individual mice is an indication of the overall health of an animal and is used as an endpoint in the mouse lethal challenge influenza model.

### Procedure:

Mice were individually weighed prior to treatment and then every day thereafter until day 21 post virus exposure or until death of the animal to assess the effects of each treatment on ameliorating weight loss due to virus infection.

### Results:

The data are summarized in Table 5.

TABLE 5
Animal weight loss as percent of initial weight (about 19 g) on Day 6
after viral infection and treatment with beraprost isomers A to D,
beraprost and placebo.

| Compound | Average % loss | Significance vs PSS |
|---|---|---|
| PSS | 72 | — |
| A | 76 | P < 0.05 |
| B | 74 | NS |
| C | 71 | NS |
| D | 68 | NS |
| Beraprost | 76 | P < 0.05 |
| Ribavirin | 89 | P < 0.005 |

NS = Not significant

### Example 6

### Demonstration of Superiority of Isomer A of Beraprost in a Mouse Lethal Challenge Model of Influenza

### Day 6 Lung Weight and Score

### Principle:

The lung weight and lung score are sensitive methods to determine the current condition of the lung. Upon infection, cells enter the lungs and the lungs fill with fluid. Therefore, the inflammation status of the lung can be determined using lung weight. The higher the weight, the greater the inflammation.

### Procedure:

At day 6, five mice from each group were humanely euthanized to harvest lungs for lung weight and lung score determination. Each mouse lung lobe was removed, weighed, placed in a petri dish, and then assigned a score ranging from 0 (normal appearing lung) to 4 (maximal plum coloration in 100% of lung).

Significant lung score differences between treatment groups were determined using a Kruskal-Wallis test, followed by Dunn's posttest for evaluating significant pairwise comparisons. Significant lung weight differences compared to the placebo-treated mice were evaluated by analysis of variance, after which individual treatment values were compared to the PSS control using a Newman-Keuls pair-wise comparison test.

### Results:

The lung weight and lung score obtained at day 6 after viral infection are listed in Table 6 and 7.

TABLE 6
Lung weight on Day 6 after viral infection and treatment with the different
isomers of beraprost and beraprost compared to placebo-treated mice.

| Compound | Mean (grams) | SD |
|---|---|---|
| PSS | 0.35 | 0.02 |
| A | 0.22 | 0.03 |
| B | 0.36 | 0.03 |
| C | 0.31 | 0.04 |
| D | 0.37 | 0.05 |
| Beraprost | 0.24 | 0.08 |
| Ribavirin | 0.16 | 0.01 |

TABLE 7
Lung score on Day 6 after viral infection and treatment with the different
isomers of beraprost and beraprost compared to placebo-treated mice.

| Compound | Mean | SD |
|---|---|---|
| PSS | 2.88 | 0.48 |
| A | 2.00 | 0.41 |
| B | 3.33 | 0.29 |
| C | 3.25 | 0.29 |
| D | 3.50 | 0.41 |
| Beraprost | 2.0 | 0.71 |
| Ribavirin | 0.00 | 0.00 |

### Example 7

### Demonstration of Superiority of Isomer A of Beraprost in a Mouse Lethal Challenge Model of Influenza

### Decreased Cell Infiltrates

### Principle:

Another measurement of the inflammatory status of the lung is to count the number of inflammatory cells in the lung. Treatment with a compound that reduces lung inflammation by modulating the immune response will reduce the number of infiltrated cells in the lung.

### Procedure:

Mouse lung cells were isolated using the following protocol. One half of the lung tissue of each mouse was excised and homogenized by wrapping the tissue in plastic sheet then rolled back and forth with a 10 mL pipette. 2 mL of cold DMEM culture media was added and the homogenate was collected into a 15 mL conical tube.

The homogenate was centrifuged at 400×g for 2 min. 0.5 mL of supernatant was collected and then further centrifuged at 1000×g at room temperature for 5 min. The clarified supernatant was collected for quantification of cytokines using multiplex immunoassay.

### Results:

The results are shown in Table 8.

TABLE 8
Mean number of cells at Day 6 after viral infection and treatment with the
isomers of beraprost and beraprost compared to placebo-treated mice

| Group | Compound | Mean | SD | SEM |
|---|---|---|---|---|
| 1 | PSS | 3.42 | 0.81 | 0.41 |
| 3 | A | 1.15 | 0.40 | 0.18 |
| 5 | B | 4.77 | 0.60 | 0.35 |
| 7 | C | 3.70 | 1.09 | 0.49 |
| 11 | Beraprost sodium | 2.63 | 1.78 | 0.80 |
| 13 | Ribovirin | 1.67 | 0.69 | 0.31 |

### Example 8

### Demonstration of Superiority of Isomer A of Beraprost in a Mouse Lethal Challenge Model of Influenza

### Decreased Cytokine Release

### Principle:

The endpoint for a treatment which modulates the immune system is a reduction in the level of pro-inflammatory cytokines in the lung.

### Procedure:

In the mouse lethal challenge influenza model (GEM-12SBIR-2), one of the harvested lungs was treated and the level of specific mouse cytokines in the resulting supernatant was measured in pg/mL using ELISA.

### Results:

The results for pro-inflammatory cytokines at day 6 for the individual isomers of beraprost, beraprost and placebo treated mice is shown in Table 9. As a determination that not all cytokines are reduced, the concentration (pg/mL) in the lung of cytokine IL-12 is shown in Table 10.

TABLE 9
Lung cytokine concentration (pg/mL) in mice treated with beraprost,
individual isomers of beraprost and placebo treated mice at day 6
after viral administration.

| Com- | CCL2 | CCL2 STD | IFNg | IFNg STD |
|---|---|---|---|---|
| pound | Average | deviation | Average | deviation |
| PSS | 2279 | 175 | 2187 | 275 |
| Cmp A | 953 | 252 | 1046 | 370 |
| Cmp B | 2365 | 82 | 2221 | 187 |
| Cmp C | 2183 | 400 | 2017 | 288 |
| Cmp D | 2347 | 270 | 2074 | 202 |
| Beraprost | 1308 | 448 | 1601 | 331 |
| Ribavirin | 304 | 32 | 531 | 54 |
| Uninfect | 17 | 1 | 1 | 1 |
| Com- | IL-6 | IL-6 STD | IL-10 | IL-10 STD |
| pound | Average | deviation | Average | deviation |
| PSS | 1109 | 354 | 868 | 129 |
| Cmp A | 521 | 271 | 223 | 116 |
| Cmp B | 1001 | 390 | 840 | 87 |
| Cmp C | 816 | 462 | 838 | 278 |
| Cmp D | 1291 | 152 | 758 | 211 |
| Beraprost | 604 | 433 | 621 | 108 |
| Ribavirin | 98 | 20 | 96 | 19 |
| Uninfect | | | 9 | 5 |

TABLE 10
Lung cytokine concentration (pg/mL) in mice treated with beraprost,
individual isomers of beraprost and placebo treated mice at day 6
after viral administration.

| | | IL-12 STD |
|---|---|---|
| Compound | IL-12 Ave | deviation |
| PSS | 3306 | 445 |
| Cmp A | 3800 | 836 |
| Cmp B | 3497 | 1373 |
| Cmp C | 3856 | 702 |
| Cmp D | 2603 | 176 |
| Beraprost | 3517 | 741 |
| Ribavirin | 1371 | 274 |
| Uninfect | 221 | 31 |

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A method of treating a viral disease associated with the induction of immune response comprised of large amounts of pro-inflammatory cytokines such as IFN-γ, IL-10, IL-6, and CCL2, a ‘cytokine storm’, in a subject in need of such treatment, said method comprising administering, or causing to be administered, to the subject amount of a therapeutic agent effective to partially or fully suppress said cytokine storm.

2. The method of claim 1, wherein said viral disease was initiated by an infection with the influenza A virus.

3. The method of claim 2, wherein the influenza A virus is H5N1 or a mutation thereof.

4. The method of claim 1, wherein said viral disease is a disease initiated by a coronavirus, for example the virus which cause the severe acute respiratory syndrome (SARS) or mutations thereof.

5. The method of claim 1, wherein said viral disease is influenza A virus.

6. The method of claim 1, wherein said viral disease is not influenza virus.

7. The method of claim 6, wherein said disease initiated by an infection with a virus selected from the group consisting of Hepatitis A virus, Hepatitis B virus, and Hepatitis C virus.

8. The method of claim 6, wherein said disease is a disease initiated by an infection with a virus selected from the group consisting of a coronavirus, Dengue virus, and West Nile Virus.

9. The method of claim 8, wherein said the virus is the virus that causes severe acute respiratory syndrome (SARS).

10. The method of claim 1, wherein said therapeutic agent comprises predominantly no more than two isomers of beraprost.

11. The method of claim 10, wherein said therapeutic agent comprises predominantly a single isomer of beraprost.

12. The method of claim 10, wherein said therapeutic agent comprises a substantially pure isomer of beraprost.

13. The method of claim 10, wherein said isomer comprises Beraprost, (2,3,3a,8b-tetrahydro-2-hydroxyl-1-(3-hydroxyl-4-methyl-1-octen-6-ynyl)-1H-cyclopenta[b]benzofuran-5-butanoic acid, sodium salt).

14. The method of claim 10, wherein said isomer comprises, wherein said the beraprost isomer BPS-314d [1R,2R,3aS, 8bS]-(2,3,3a,8b-tetrahydro-2-hydroxyl-1-[(3S,4S)-(3-hydroxyl-4-methyl-1-(E)-octen-6-ynyl)-1H-cyclopenta[b]benzofuran-5-butanoic acid.

15. The method of claim 1, wherein said agent is administered via a route selected from the group consisting of inhalation, transdermal, intravenous, subcutaneous, and oral administration.

16. The method of claim 15, wherein said agent is administered in a therapeutically effective amount ranging from about 0.050 mg/day to 1 mg/day.

17. A method of treating a viral disease that induces a cytokine storm in an individual, said method comprising administering to said individual a therapeutically effective amount of a prostacyclin analog.

18. A therapeutic composition comprising a therapeutic agent wherein said therapeutic agent comprises predominantly no more than two isomers of beraprost.

19. The composition of claim 18, wherein said therapeutic agent comprises predominantly a single isomer of beraprost.

20. The composition of claim 18, wherein said therapeutic agent comprises a substantially pure isomer of beraprost.

21. The composition of claim 18, wherein said isomer comprises Beraprost, (2,3,3a,8b-tetrahydro-2-hydroxyl-1-(3-hydroxyl-4-methyl-1-octen-6-ynyl)-1H-cyclopenta[b]benzofuran-5-butanoic acid, sodium salt).

22. The composition of claim 18, wherein said isomer comprises, wherein said the beraprost isomer BPS-314d [1R,2R,3aS, 8bS]-(2,3,3a, 8b-tetrahydro-2-hydroxyl-1-[(3S,4S)-(3-hydroxyl-4-methyl-1-(E)-octen-6-ynyl)-1H-cyclopenta[b]benzofuran-5-butanoic acid.

23. The composition of claim 18, wherein said agent formulated for administration via a route selected from the group consisting of inhalation, transdermal, intravenous, subcutaneous, and oral administration.

24. The composition of claim 23, wherein said composition is a unit dosage formulation.
